(19) United States
(12) Patent Application Publication
(10) Pub. No.: US 2025/0114571 A1
(43) Pub. Date: Apr. 10, 2025

(54) INTRODUCER SHEATH FOR INTRODUCING INTERVENTION DEVICES INTO VASCULAR SYSTEMS

(71) Applicant: Contract Medical International GmbH, Dresden (DE)

(72) Inventor: Tobias ROKOSCH, Dresden (DE)

(21) Appl. No.: 18/906,977

(22) Filed: Oct. 4, 2024

(30) Foreign Application Priority Data
Oct. 10, 2023 (DE) 102023127557.7

(51) Int. Cl. A61M 25/06; A61M 25/00

(52) U.S. Cl. CPC A61M 25/0662; A61M 25/0009; A61M 25/0045; A61M 25/005; A61M 2025/0047; A61M 2205/0216; A61M 2207/10

(57) ABSTRACT

An introducer sheath for introducing intervention devices into vascular systems, comprising an introducer sheath body comprising a proximal end, a distal end, and a cavity that extends between the proximal end and the distal end, and an inner lining, an outer casing, and a reinforcing layer, arranged between the inner lining and the outer casing.

### CROSS-REFERENCE TO RELATED APPLICATIONS

This application claims priority pursuant to 35 U.S.C. 119(a) to German Application No. 102023127557.7, filed Oct. 10, 2023, which application is incorporated herein by reference in its entirety.

### FIELD OF THE INVENTION

The invention relates to an introducer sheath for introducing intervention devices into vascular systems, comprising:
an introducer sheath body comprising a proximal end, a distal end, and a cavity that extends between the proximal end and the distal end,
and an inner lining, an outer casing, and a reinforcing layer, arranged between the inner lining and the outer casing.

The invention also relates to a method for manufacturing such an introducer sheath.

### BACKGROUND OF THE INVENTION

Among the most significant medical and surgical advances in recent decades has been the introduction and now routine implementation of a large number of minimally invasive procedures. These procedures include angioplasty, endoscopy, laparoscopy, and arthroscopy along with numerous other diagnostic and therapeutic interventions. Such minimally invasive procedures differ from conventional open surgical procedures in that access to a specific site in the patient is gained via a small incision into which a tubular device, or at least part of it, is inserted or introduced. The tubular device, or the tubular part of the device, keeps the incision open and at the same time enables access to the vascular system of the patient via a cavity inside the device. A particularly significant example of a minimally invasive technique is the temporary or permanent implantation of a medical intervention device, such as a stent, into a vessel of a patient.

Other examples relate to the transfer of a liquid medication to a target area and/or the removal of body fluid from the vascular system.

Introducer sheaths are typically used when performing these and other desired techniques—particularly when introducing intervention devices.

However, introducer sheaths tend to kink—in particular, when introduced into angled parts of the vascular system of the patient. Kinking reduces the effective inner diameter of the introducer sheath, which often makes it unusable. At the same time, the introducer sheath must be highly flexible—in particular, in order to be able to be introduced into the narrower vessels of the patient.

In order to be able to be introduced into narrower vessels and at the same time ensure the largest possible cavity for the feeding through of intervention devices, introducer sheaths often only have thin walls, wherein the wall is equipped with a reinforcing layer for structural stabilization in order to achieve high resistance to kinking at the same time. Such an introducer sheath is described, for example, in U.S. Pat. No. 5,700,253.

However, such reinforcing layers should not be exposed on the inner side of the introducer sheath, since this would make it more difficult for intervention devices to feed through the cavity of the introducer sheath—for example, due to increasing friction or getting stuck. Therefore, the inside of the introducer sheath usually has a polymer inner lining, which is intended to enable intervention devices to feed through the introducer sheath as easily and smoothly as possible. The inner lining should have the lowest possible coefficient of friction in order to enable such devices to be introduced smoothly through the cavity of the introducer sheath. Polytetrafluoroethylene-based (PTFE) coatings, such as those described in EP 1 853 311 B1, predominate on the market.

However, due to the potential environmental impact of PTFE materials, there is a desire in the market for a suitable replacement for PTFE-based inner linings.

### SUMMARY OF THE INVENTION

A contribution to the at least partial fulfillment of at least one of the below-mentioned objects is made by the features of the independent claims. The dependent claims provide preferred embodiments that contribute to at least partial fulfillment of at least one of the objects.

A first embodiment of the invention is an introducer sheath for introducing intervention devices into vascular systems of a patient, comprising an introducer sheath body comprising a proximal end, a distal end, and a cavity that extends between the proximal end and the distal end, wherein the introducer sheath body has an inner lining, an outer casing, and reinforcing layer arranged between the inner lining and the outer casing, wherein the inner liner consists of a material comprising a thermoplastic elastomer and a siloxane, wherein the elastomer has a weight percentage in a range of 90-99 percent by weight, and the siloxane has a weight percentage in a range of 1-10 percent by weight, in each case relative to the total weight of the material.

In one embodiment of the introducer sheath, the elastomer is a polyether block amide copolymer (PEBA). This embodiment is a second embodiment of the invention, which is preferably dependent upon the first embodiment of the invention.

In one embodiment of the introducer sheath, the polyether block amide copolymer has a melting point in a range of 130-175° C., preferably in a range of 150-175° C., more preferably in a range of 165-175° C. This embodiment is a third embodiment of the invention, which is preferably dependent upon the second embodiment of the invention.

In one embodiment of the introducer sheath, the siloxane has a molecular weight in a range of 500-1,500 kDa (kilodaltons), preferably in a range of 700-1,300 kDa, more preferably in a range of 800-1,200 kDa. This embodiment is a fourth embodiment of the invention, which preferably depends upon one of the preceding embodiments of the invention.

In one embodiment of the introducer sheath, a siloxane concentration in the material is higher on an outer surface of the inner liner—in particular, on an outer surface, facing the cavity of the introducer sheath body, of the inner liner—than in the core of the inner liner. This embodiment is a fifth embodiment of the invention, which preferably depends upon one of the preceding embodiments of the invention.

In one embodiment of the introducer sheath, the inner lining is substantially free of fluorine compounds—in particular, free of polytetrafluoroethylene-containing compounds. This embodiment is a sixth embodiment of the invention, which preferably depends upon one of the preceding embodiments of the invention.

In one embodiment of the introducer sheath, the inner lining has an inner diameter in a range of 1.3-12 mm—preferably in a range of 1.3-3 mm. This embodiment is a seventh embodiment of the invention, which preferably depends upon one of the preceding embodiments of the invention.

In one embodiment of the introducer sheath, the inner lining has a wall thickness in a range of 0.05-0.5 mm—preferably in a range of 0.1-0.2 mm. This embodiment is an eighth embodiment of the invention, which preferably depends upon one of the preceding embodiments of the invention.

In one embodiment of the introducer sheath, the outer casing comprises a polyether block amide copolymer. This embodiment is a ninth embodiment of the invention, which preferably depends upon one of the preceding embodiments of the invention.

In one embodiment of the introducer sheath, the outer casing has a wall thickness in a range of 0.1-0.5 mm—preferably in a range of 0.1-0.2 mm. This embodiment is a tenth embodiment of the invention, which preferably depends upon one of the preceding embodiments of the invention.

In one embodiment of the introducer sheath, the reinforcing layer comprises a braided metal band, a spirally wound, i.e., spiral-spring-like, metal band. This embodiment is an eleventh embodiment of the invention, which preferably depends upon one of the preceding embodiments of the invention.

In one embodiment of the introducer sheath, the metal strip has a thickness in a range of 0.2-0.5 mm. This embodiment is a twelfth embodiment of the invention, which is preferably dependent upon the eleventh embodiment of the invention.

A thirteenth embodiment of the invention is a method for manufacturing an introducer sheath according to one of the preceding embodiments of the invention, comprising the following method steps:
a. providing the inner lining, the outer casing, and the reinforcing layer;
b. arranging the inner lining around a mandrel;
c. arranging the reinforcing layer around the inner lining;
d. arranging the outer casing around the reinforcing layer;
e. encasing the outer casing with a shrink tube to form a structure;
f. heating the structure comprising the inner lining, the reinforcing layer, the outer casing, and the shrink tube while shrinking the shrink tube and while joining with a material bond the inner lining to the outer casing by means of the reinforcing layer;
g. removing the mandrel.

In one embodiment of the method, the provision of the inner liner comprises extruding a granulate mixture comprising the thermoplastic elastomer and the siloxane. This embodiment is a fourteenth embodiment of the invention, which is preferably dependent upon the thirteenth embodiment of the invention.

In one embodiment of the method, the granulate mixture comprises a first granulate consisting of the thermoplastic elastomer and a second granulate comprising the siloxane in a matrix of the thermoplastic elastomer. This embodiment is a fifteenth embodiment of the invention, which is preferably dependent upon the fourteenth embodiment of the invention.

One object of the present invention is to overcome, at least in part, one or more of the disadvantages resulting from the prior art.

In particular, the invention is based upon the objective of providing an introducer sheath—in particular, an inner lining of an introducer sheath—which enables intervention devices to feed through as smoothly, easily, and in as trouble-free a manner as possible. Furthermore, the introducer sheath should be designed to be as flexible as possible, such that introduction is enabled even in narrower and more angled vascular systems of a patient. The introducer sheath should comprise materials that are as environmentally compatible as possible; in particular, the introducer sheath should be constructed free of materials containing polytetrafluoroethylene. The material of the inner lining of the introducer sheath should be compatible with the biocompatible materials commonly used on the market for conventional introducer sheaths, in order to be able to use standard materials and processes. It should be possible to provide the inner lining with the simplest possible processes. The introducer sheath should be mechanically robust.

It is a further object of the invention to provide a method for manufacturing such an introducer sheath, by means of which at least some of the objects already described are at least partially achieved.

### GENERAL

In the present description, specifications of ranges also contain the values specified as limits. A specification of the type “in the range from X to Y” with respect to a quantity A consequently means that A can take the values X, Y and values between X and Y. One-sidedly limited ranges of the type “up to Y” for a size A accordingly mean as a value Y and less than Y.

Some of the features described are associated with the term “substantially.” The term “substantially” is to be understood in such a way that, under real conditions and manufacturing techniques, a mathematically exact interpretation of terms such as “superimposition,” “perpendicular,” “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastic” comprises a plastic content of >95 to <100% by weight. For example, a “substantially complete filling of a volume B” comprises a filling of >95 to <100% by volume of the total volume of B.

Terms such as “proximal” and “distal” merely serve to designate the spatially opposite ends of the device or other structural units of the device, and do not allow any conclusions to be drawn about the orientation in relation to a human body—for example, a user of the device. “Distal to . . . ” and “proximal to . . . ” or similar formulations merely express the spatial arrangement of two structural units of the device in relation to one another.

### DETAILED DESCRIPTION

A first subject matter of the invention relates to an introducer sheath for introducing intervention devices into vascular systems of a patient, comprising an introducer sheath body comprising a proximal end, a distal end, and a cavity that extends between the proximal end and the distal end, wherein the introducer sheath body has an inner lining, an outer casing, and a reinforcing layer arranged between the inner lining and the outer casing, wherein the inner liner consists of a material comprising a thermoplastic elastomer and a siloxane, wherein the elastomer has a weight percentage in a range of 90-99 percent by weight, and the siloxane has a weight percentage in a range of 1-10 percent by weight, in each case relative to the total weight of the material.

The introducer sheath comprises a tube-like introducer sheath body, which has at least three, coaxially superimposed layers. A cavity extends axially through the introducer sheath body from a proximal end to a distal end, which cavity is designed for introducing intervention devices into a vascular system of a patient. The cavity is encased by the three layers of the introducer sheath body, wherein the three layers comprise, from the inside to the outside, an inner lining, a reinforcing layer, and an outer casing. In other words, the cavity is radially surrounded by the inner lining, the inner lining is radially surrounded by the reinforcing layer, and this in turn is radially surrounded by the outer casing. Preferably, there are no other layers or air pockets between the inner lining and the reinforcing layer, or between the reinforcing layer and the outer casing.

Each of these layers has, in itself, a tube-like shape.

The reinforcing layer preferably has feedthroughs, such that the inner lining can form a joining with a material bond with the outer casing, during the manufacture of the introducer sheath body, by applying heat. The reinforcing layer is thus preferably firmly enclosed by the inner lining and the outer casing, and is substantially located in the region of the interface between the inner lining and the outer casing within the manufacturing tolerances.

The inner lining faces the tunnel-like cavity in the interior of the introducer sheath—in particular, the introducer sheath body. Thus, the cavity is formed by an outer surface, facing the longitudinal axis of the introducer sheath body, of the inner lining.

Thus, the inner lining comes into direct contact with intervention devices, such as catheters or stents, which are introduced into the vascular system of a patient.

In order to make the introducer sheath—in particular, the introducer sheath body—flexible, and thus enable the introducer sheath to be introduced even into angled parts of the vascular system, the inner lining consists of a material that consists of 90-99 percent by weight of a thermoplastic elastomer relative to the total weight of the material. The thermoplastic elastomer can consist of a pure thermoplastic elastomer or a mixture of several, e.g., two or three, different thermoplastic elastomers. Preferably, the thermoplastic elastomer consists of a pure thermoplastic elastomer.

Thermoplastic elastomers have the advantage that they are usually easier to process than the standard polytetrafluoroethylene-containing materials on the market, thus enabling simplified manufacturing, as well as being more flexible than these, with otherwise identical dimensions.

In order to reduce the coefficient of friction of the thermoplastic elastomer and thus facilitate the introduction of intervention devices through the cavity, the material of the inner lining comprises 1-10 percent by weight, preferably 2-8 percent by weight, more preferably 3-6 percent by weight, in each case relative to the total weight of the material, of siloxane. The siloxane can consist of a pure siloxane or a mixture of several, e.g., two or three, different siloxanes. Preferably, the siloxane consists of a pure siloxane. If the material contains less than the specified amounts of siloxane—in particular, less than 1 percent by weight—the coefficient of friction of the material is not reduced sufficiently to ensure smooth introduction of an intervention device through the cavity. If the material contains more than the specified amounts—in particular, more than 10 percent by weight, compatibility with the outer casing is reduced, which increases the risk of delamination between the inner lining and the outer casing.

In addition to the introducer sheath body, the introducer sheath can comprise other components, such as a hemostatic valve, an internal dilator, a Luer adapter with a lateral connection for flushing, and/or an infusion access on the hemostatic valve.

Various thermoplastic elastomers can be used as thermoplastic elastomers in pure form or as a mixture of several different thermoplastic elastomers. Preferably, the thermoplastic elastomers are biocompatible. Examples of thermoplastic elastomers comprise polyesters, polyurethanes, polyolefins, and styrenes.

One embodiment of the introducer sheath is characterized in that the thermoplastic elastomer is a polyether block amide copolymer (PEBA). Polyether block amide copolymers are available, for example, under the brand name, PEBAX®, from Arkema S.A., France, or the brand name, VESTAMID® E, from Evonik Industries AG, Germany. Polyether block amide copolymers have the advantage of being easy to process and biocompatible. They are also characterized by good mechanical properties, such as their flexibility, and are available in a wide range from soft to firm.

Depending upon the application, polyether block amide copolymers with different material properties, such as different melting points, can be used.

One embodiment of the introducer sheath is characterized in that the polyether block amide copolymer has a melting point in a range of 130-175° C.—preferably in a range of 165-172° C. The higher the melting point, the lower the flexibility and the higher the resistance to kinking. The specified melting point range offers a good compromise between flexibility and resistance to kinking of the inner lining.

The siloxanes used in the material of the inner lining can have different material properties. For example, the siloxanes can be functionalized with different organic functional groups—in particular, alkyl groups. In particular, the organic functional groups can be matched to the thermoplastic elastomer to be used—for example, to improve compatibility with it. For example, the siloxanes can be functionalized with methyl groups.

Siloxanes with different molecular weights can also be used, to achieve the desired reduction in the coefficient of friction of the inner lining.

In one embodiment of the introducer sheath, the siloxane has a molecular weight in a range of 500-1,500 kDa (kilodaltons), preferably in a range of 700-1,300 kDa, more preferably in a range of 800-122 kDa. Such types of siloxanes are sometimes also referred to as ultra-high molecular weight (UHMW) siloxanes. Although the ability to process the siloxane becomes more difficult with increasing molecular weight, the effectiveness of the reduction in the coefficient of friction per weight percentage added increases at the same time. Thus, the specified range represents a good compromise between ability to process and effectiveness in reducing the coefficient of friction.

In one embodiment of the introducer sheath, a siloxane concentration in the material on an outer surface of the inner lining—in particular, an outer surface, facing the cavity, of the inner lining—is higher than in the core of the inner lining. Thus, the material exhibits a gradient in siloxane concentration, wherein the concentration of siloxane is highest on the outer surface. The increased siloxane concentration on the outer surface can be achieved by functionalizing the siloxane as a function of the thermoplastic elastomer used and/or by using siloxanes with an increased molecular weight, in particular in a range of 500-1,500 kDa (kilodaltons), preferably in a range of 700-1,300 kDa.

This has the advantage that a smaller amount of siloxane is required to achieve the same, reduced coefficient of friction in the material. This can, for example, facilitate the ability to process the material.

In one embodiment of the introducer sheath the inner lining is substantially free of fluorine compounds—in particular, free of polytetrafluoroethylene-containing materials. Preferably, no fluorine-containing compound—in particular, no polytetrafluoroethylene-containing material—is added to the material.

An inner diameter of the inner lining, which preferably corresponds to an outer diameter of the cavity, can assume different values depending upon the field of application of the introducer sheath.

In one embodiment of the introducer sheath the inner lining has an inner diameter in a range of 1.3-12 mm, preferably in a range of 1.3-3 mm.

In principle, with introducer sheaths, one tries to achieve the lowest possible wall thickness of the introducer sheath body. In this way, a cavity with the largest possible outer diameter can be provided for a given outer diameter of the introducer sheath body, and the available diameter of the vascular system can be utilized in the best possible way for the introduction of intervention devices.

In one embodiment of the introducer sheath the inner lining has a wall thickness—in other words, a thickness—in a range of 0.05-0.5 mm, preferably in a range of 0.1-0.2 mm. Greater wall thicknesses would excessively restrict the diameter available for the feeding through of intervention devices through the cavity for the given outer diameter of the introducer sheath body. Smaller wall thicknesses—in particular, when using polyether block amide copolymers as thermoplastic elastomers—increase the risk of a reinforcing layer being exposed in the cavity—at least in sections. This risk exists, since the introducer sheath is preferably manufactured by joining with a material bond the inner lining to the outer casing, i.e., by fusing. During fusion—in particular, of polyether block amide copolymers—the reinforcing layer can be unintentionally exposed—especially if the wall thickness is less than 0.1 mm. This increases the coefficient of friction at the corresponding point. This can also lead to the intervention device fed through the cavity jamming on the exposed reinforcing layer.

The outer casing can comprise different materials or consist of different materials. Preferably, the outer casing, like the inner lining, comprises a thermoplastic elastomer or consists of a thermoplastic elastomer. Since it is preferable to manufacture the introducer sheath body by joining with a material bond the inner lining and outer casing, the materials of the inner lining and outer casing should be compatible wherever possible. If the materials are incompatible, a joining layer can be applied to the outer side of the inner lining and/or the inner side of the outer lining, which enables a joining with a material bond of the two layers when heated. Preferably, such a joining layer is applied to the outer side of the inner lining. Such a joining layer is sometimes referred to as a “tie layer.” In addition to one or more joining layers, an outer surface of the corresponding layers can be treated to improve the joining between the inner lining and the outer casing. For example, the outer surface of the inner lining can be etched or treated with plasma to increase its surface area.

In one embodiment of the introducer sheath the outer casing comprises a polyether block amide copolymer or consists of a polyether block amide copolymer. In the preferred case that the inner lining also comprises a polyether block amide copolymer, this enables the best possible compatibility both in the manufacture of the introducer sheath and in the mechanical properties of the two layers. This reduces the risk of delamination of the two layers—in particular, when bending the introducer sheath. Preferably, both layers comprise the same polyether block amide copolymer. This is also advantageous during manufacture, since both layers have the same or at least similar temperature behavior—in particular, the same or at least similar melting point.

The outer casing can have different wall thicknesses, depending upon the application of the introducer sheath.

In one embodiment of the introducer sheath the outer casing has a wall thickness in a range of 0.1-0.5 mm, preferably in a range of 0.1-0.2 mm. For a given outer diameter of the cavity, this enables the smallest possible outer diameter of the introducer sheath body. In addition, this wall thickness range is within the preferred wall thickness range of the inner lining, which means that the mechanical properties of these two layers are similar—in particular, if the material properties are the same or similar. This can reduce the risk of delamination of the two layers—in particular, when bending.

Furthermore, a wall thickness of the outer casing of at least 0.1 mm reduces the risk of the reinforcing layer being unintentionally exposed during the manufacture of the introducer sheath—in particular, during the preferred joining with a material bond of the inner lining to the outer casing. An externally exposed reinforcing layer can lead to vascular injury when the introducer sheath is introduced into the vascular system of a patient.

The reinforcing layer reduces the risk of kinking of the introducer sheath—in particular, the introducer sheath body. Preferably, the reinforcing layer has feedthroughs such that the inner lining can form a joining with a material bond with the outer casing by applying heat during the manufacture of the introducer sheath body. The reinforcing layer is thus preferably firmly enclosed by the inner lining and the outer casing, and is substantially located in the region of the interface between the inner lining and the outer casing within the manufacturing tolerances.

The reinforcing layer can comprise different materials or consist of different materials. Preferably, the reinforcing layer comprises a polymer, a metal, or consists of a polymer or a metal. Examples of suitable polymers are polyether ether ketones (PEEK). Examples of suitable metals are titanium and alloys such as nickel-titanium alloys and stainless steel—in particular, 304V or 3161 stainless steel.

In one embodiment, the reinforcing layer comprises a braided wire—in particular, a round wire—and/or a spiral wire—in particular, a round wire—preferably a braided metal wire and/or a spiral metal wire, or the reinforcing layer consists of such a wire—in particular, a round wire.

In one embodiment of the introducer sheath the reinforcing layer comprises a braided metal strip and/or a spiral metal strip, or the reinforcing layer consists of such a metal strip. One advantage of a metal strip is that it allows the reinforcing layer to be constituted by the least possible thickness, while retaining its function.

In one embodiment of the introducer sheath is characterized in that the metal strip has a thickness in a range of 0.2-0.5 mm.

A further subject matter of the invention relates to a method for manufacturing an introducer sheath according to one of the preceding embodiments of the invention, comprising the following method steps:
a. providing the inner lining, the outer casing, and the reinforcing layer;
b. arranging the inner lining around a mandrel;
c. arranging the reinforcing layer around the inner lining;
d. arranging the outer casing around the reinforcing layer;
e. encasing the outer casing with a shrink tube to form a structure;
f. heating the structure comprising the inner lining, the reinforcing layer, the outer casing, and the shrink tube while shrinking the shrink tube and while joining with a material bond the inner lining to the outer casing by means of the reinforcing layer;
g. removing the mandrel.

The inner lining, the outer casing, and the reinforcing layer can be provided in different ways in method step a. In one embodiment of the method, all three layers of the introducer sheath body can be provided in a length that substantially corresponds to the planned length of the final introducer sheath body. In a further embodiment of the method, the three layers can be provided with a length of a multiple of the planned length of the final introducer sheath body, such that the final introducer sheath body can be obtained by cutting to the desired length. Preferably, however, all three layers are substantially the same length.

In method steps b., c., and d., the inner lining, the reinforcing layer, and the outer casing are layered on top of one another on a mandrel. The inner lining is located radially on the inside, the outer casing radially on the outside, and the reinforcing layer between the inner lining and the outer casing. Further layers, such as a joining layer (tie layer), can be provided between the individual layers, wherein it is preferable for the three layers to be arranged directly on top of one another.

In method step e., the at least three layers are encased with a shrink tube. A shrink tube is a tube that contracts radially when exposed to heat, thereby exerting a pressure acting radially inwards. Shrink tubing suitable for the method according to the invention is made of a fluoroethylene propylene (FEP), for example. By encasing with the shrink tube, a structure is created comprising at least three layers of the introducer sheath body, along with the outer shrink tube and the inner mandrel.

In step f., the structure is heated such that the shrink tube shrinks, thereby exerting a pressure acting radially inwards upon the at least three layers of the introducer sheath body to be manufactured. The internal mandrel limits the at least three layers radially inwards, such that they are pressed together between the shrink tube and mandrel. The heat input causes at least the inner lining and the outer casing to melt at the same time, such that the two layers preferably form a joining with a material bond through the feedthroughs in the reinforcing layer, and a stable introducer sheath body is formed. The temperature to which the structure is heated during method step f. depends upon the materials used, but is preferably in a temperature range between 170-230 C°—in particular, when polyether block amide copolymer is used as the thermoplastic elastomer. In method step f., the introducer sheath body of the introducer sheath is thus formed.

In method step g., the introducer sheath body is removed from the mandrel. As an option, the length of the introducer sheath body can be adapted by cutting and/or removing the shrunken shrink tube—for example, by cutting or peeling it off.

In one embodiment of the method, the inner lining is provided by extruding a granulate mixture comprising the thermoplastic elastomer and the siloxane. A granulate is a substance consisting, in the broadest sense, of many small, solid particles such as grains, balls, or spheres. Such a substance can sometimes also be referred to as a powder—especially in the case of particularly small particles. Within the granulate mixture, the thermoplastic elastomer with the siloxane can be present as compounded granulate.

In one embodiment of the method, the granulate mixture comprises a first granulate consisting of the thermoplastic elastomer and a second granulate comprising the siloxane in a matrix of the thermoplastic elastomer. In this embodiment, the siloxane is present in increased concentration—preferably between 10-50 percent by weight relative to the total weight of the second granulate—dissolved, or dispersed in the same thermoplastic elastomer with which the material of the inner lining is later manufactured. This facilitates the manufacture of inner linings with different weight proportions of thermoplastic elastomer to siloxane. The use of the same thermoplastic elastomer in the first and second granules ensures good compatibility of the granules during the manufacturing process.

The features disclosed for the introducer sheath are also disclosed for the method, and vice versa.

### EXAMPLES

The invention is further illustrated below using examples which are, however, not to be understood as limiting. It will be apparent to a person skilled in the art that other equivalent means may be used similarly in place of the features described here.

### Manufacturing Method

A granulate of a polyether block amide copolymer (PEBAX® 6333 SAOmed, Arkema S.A., France) and, if applicable, a siloxane (EverGlide® MED, Polymer Dynamix, USA) were used to manufacture the inner linings of Examples 1 and 2 according to the invention and Comparative Examples 2 and 3 according to the proportions in Table 1. Before extruding the inner lining, the granulate was freed of residual moisture at 76° C. using a granulate dryer, an extruder was set to the final tube dimensions (inner diameter: 2.90 mm, outer diameter: 3.10 mm, cut length: 1,100 mm), and the inner lining was manufactured at 285° C. using the extruder.

An analogous method was used to manufacture the outer casing, wherein a granulate made of PEBAX® 633 SAOmed was used, and the extruder was set to final tube dimensions with an inner diameter of 3.41 mm, an outer diameter of 3.73 mm, and a cut length of 900 mm.

A reinforcing layer consisting of a stainless-steel flat wire (width: 0.40 mm, thickness: 0.076 mm) was formed into a spiral shape using a coil winding technique.

To join the individual layers, the inner lining was first pushed smoothly onto a mandrel, followed by a mounting of the reinforcing layer and then the outer casing along with, as the outermost layer, an FEP shrink tube with an inner diameter of 3.85 mm. The individual layers were bonded together by heating them to 175° C. After cooling, the shrink tube was removed, and the introducer sheath body manufactured in this way was pulled off the mandrel.

A PTFE inner lining with the dimensions of the other examples was purchased commercially for the manufacture of Comparative Example 1. In order to enable a joining with the outer casing, there was a joining layer of polyurethane on the inner lining. Manufacture was carried out in the same way as the other examples.

The inner diameter of the introducer sheath bodies of all examples was 2.75 mm.

### DESCRIPTION OF THE METHOD

A trackability test was carried out to determine the introduction force. This test simulates the introduction of a catheter through the exemplary introducer sheath bodies and determines the force to be applied. For this purpose, the exemplary introducer sheath bodies with coils, which roughly correspond to those of a femoral access, were fastened to a wall, and a catheter was introduced into them. The introducer sheaths were filled with tap water at 37° C. prior to the introduction of the catheter.

The catheter used had an outer diameter of 8 Fr and was inserted into the introducer sheaths at an introduction speed of 100 mm/min. In order to be able to recognize variances between the measurements, this was repeated three times, and the values obtained were averaged. The higher the value determined in this way, the greater the coefficient of friction of the inner lining used.

TABLE 1
Determination of the introduction force with different inner linings.

| | | | | Example 1 | Example 2 |
|---|---|---|---|---|---|
| | | | | according | according |
| | Comparative | Comparative | Comparative | to the | to the |
| | Example 1 | Example 2 | Example 3 | invention | invention |
| Material of | PTFE | PEBAX ® | 88 percent | 98 percent | 91 percent |
| the inner | | 6333 | by weight | by weight | by weight |
| lining | | SA0med | PEBAX ® | PEBAX ® | PEBAX ® |
| | | | 6333 | 6333 | 6333 |
| | | | SA0med + | SA0med + | SA0med + |
| | | | 12 percent | 2 percent | 9 percent |
| | | | by weight | by weight | by weight |
| | | | siloxane | siloxane | siloxane |
| Introduction | 196 | 265 | 196 | 216 | 206 |
| force [mN] | | | | | |
| Risk of | Low | Low | High | Low | Low |
| delamination | | | | | |
| between the | | | | | |
| inner lining | | | | | |
| and outer | | | | | |
| casing | | | | | |
| Joining layer | Yes | no | no | no | no |
| required | (polyurethane) | | | | |

The examples show that the required introduction force of inner linings made of polyether block amide copolymers can be reduced by adding siloxanes, which facilitates the introduction of devices through the introducer sheath bodies. However, increasing the addition of siloxanes increases the risk of delamination between the layers, which is why an upper limit of 10 percent by weight should not be exceeded.

### BRIEF DESCRIPTION OF THE DRAWINGS

The invention is further illustrated by way of example below by means of figures. The invention is not limited to the figures. The following are shown:

FIG. 1 is a schematic side view of an exemplary introducer sheath comprising an introducer sheath body;

FIG. 2 is a schematic cross-section of the introducer sheath body from FIG. 1; and,

FIG. 3 is a schematic side view of the introducer sheath body from FIGS. 1 and 2 in the course of its manufacture.

### DETAILED DESCRIPTION OF THE INVENTION

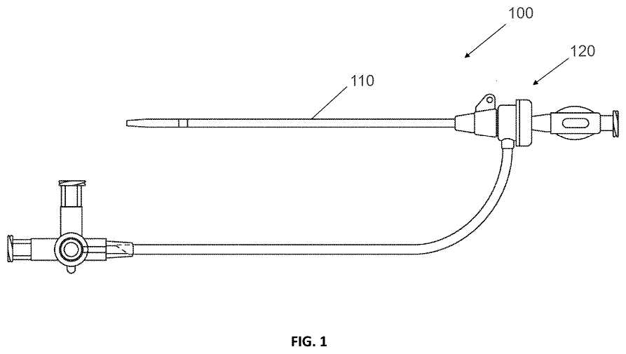

FIG. 1 shows a schematic side view of an exemplary introducer sheath 100 comprising an introducer sheath body 110, at the proximal end of which a hemostatic valve 120 is arranged.

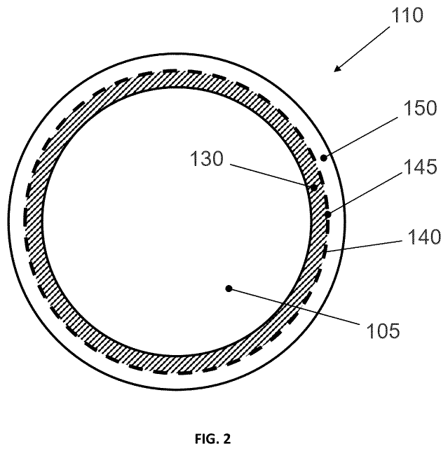

FIG. 2 shows a schematic cross-sectional view of the introducer sheath body 110 of FIG. 1.

The introducer sheath body 110 comprises an inner liner 130 that surrounds a cavity 105 inside the introducer sheath body 110. Radially outwards, the inner lining 130 is surrounded by an outer casing 150, wherein a reinforcing layer 140 is arranged between the inner lining 130 and the outer casing 150. In the embodiment shown, the introducer sheath body 110 is constructed of three layers. In further embodiments, not shown, the introducer sheath body 110 can comprise further layers, such as a joining layer between the inner liner 130 and the outer casing 150.

The inner lining 130 consists of a material that contains 95 percent by weight of a thermoplastic elastomer—in particular, a polyether block amide copolymer—and 5 percent by weight of a siloxane, in each case relative to the total weight of the material.

In the embodiment shown, the outer casing 150, like the inner lining 130, is made of a thermoplastic elastomer—in particular, a polyether block amide copolymer—in particular, the same polyether block amide copolymer. This ensures good compatibility of the inner lining 130 and the outer casing 150 during the manufacture of the introducer sheath body 110, which is achieved by a joining with a material bond by a heat input—in other words, a fusion—of the two specified layers 130, 150. To enable fusion of the inner liner 130 and the outer casing 150, the reinforcing layer 140 has a plurality of feedthroughs 145 that are distributed around the circumference and length of the reinforcing layer 140. The fusion of the inner lining 130 and the outer casing 150 can thus take place through the feedthroughs 145 of the reinforcing layer 140.

In the embodiment shown, the reinforcing layer 140 is configured as a braided metal strip—in particular, of stainless steel 304V—and is arranged substantially at an interface between the inner lining 130 and the outer casing 150.

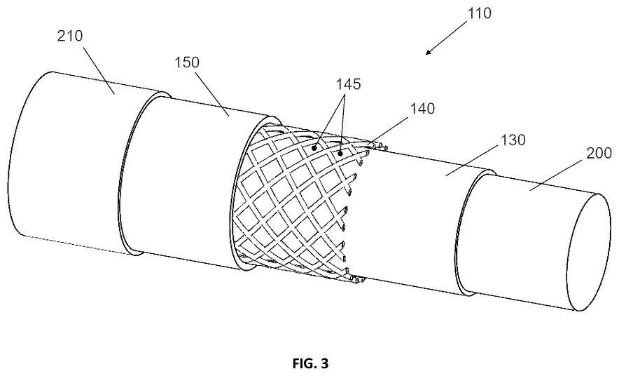

FIG. 3 shows a schematic section of the introducer sheath 110 from FIGS. 1 and 2 in a perspective side view in the course of manufacture. For a better overview of the individual layers of the superstructure, these are drawn only in sections, such that each layer can be easily recognized individually.

The three layers 130, 140, and 150 shown in FIGS. 1 and 2 are arranged on a mandrel 200 to manufacture the introducer sheath body 110. The inner lining 130 is arranged directly on the mandrel 200. The mandrel 200 ensures that the inner lining 130 does not contract during the manufacturing process. The outer diameter of the mandrel 200 thus substantially corresponds to an inner diameter of the inner lining 130, and thus also to an outer diameter of the cavity 105 (cf. FIG. 2) of the introducer sheath body 110. On the inner lining 130, the reinforcing layer 140 is arranged with a plurality of feedthroughs 145. In the view shown, it is clearly recognizable that the reinforcing layer 140 is designed as a braided metal strip, wherein the feedthroughs 145 constitute the open areas of the braid. On the reinforcing layer 140, the outer casing 150 is arranged, which, in the embodiment shown, also represents the outermost layer of the final introducer sheath body 110. On the outer casing 150, a shrink tube 210 is arranged, which radially contracts inwards, i.e., in the direction of the mandrel 200, under the input of heat. In order to join the layers 130, 140, 150 of the introducer sheath body 110, such structure is heated in such a way that the shrink tube 210 contracts and creates pressure in the direction of the mandrel 200, and that the inner lining 130 and the outer casing 150 join one another with a material bond through the feedthroughs 145 of the reinforcing layer 140. The exact temperature depends upon the materials used.

Once the inner lining 130 and outer casing 150 have been joined, the introducer sheath body 110 manufactured in this way is pulled down from the mandrel 200. Optionally, the shrink tube 210 can be removed from the outer casing 150—for example, by cutting or peeling it off.

### REFERENCE NUMBERS

100 Introducer sheath
105 Cavity
110 Introducer sheath body
120 Hemostatic valve
130 Inner lining
140 Reinforcing layer
145 Feedthroughs
150 Outer casing
200 Mandrel
210 Shrink tube

1. An introducer sheath for introducing intervention devices into vascular systems, comprising: an introducer sheath body comprising a proximal end, a distal end, and a cavity that extends between the proximal end and the distal end, wherein the introducer sheath body has an inner lining, an outer casing, and a reinforcing layer arranged between the inner lining and the outer casing, wherein the inner liner consists of a material comprising a thermoplastic elastomer and a siloxane, wherein the elastomer has a weight percentage in a range of 90-99 percent by weight, and the siloxane has a weight percentage in a range of 1-10 percent by weight, in each case relative to the total weight of the material.

2. The introducer sheath according to claim 1, wherein the elastomer is a polyether block amide copolymer.

3. The introducer sheath according to claim 2, wherein the polyether block amide copolymer has a melting point in a range of 130-175° C.—preferably in a range of 165-172° C.

4. The introducer sheath according to claim 1, wherein the siloxane has a molecular weight in a range of 500-1,500 kDa, preferably in a range of 700-1,300 kDa, more preferably in a range of 800-1,200 kDa.

5. The introducer sheath according to claim 1, wherein a siloxane concentration in the material on an outer surface of the inner liner is higher than in the core of the inner liner.

6. The introducer sheath according to claim 1, wherein the inner lining is substantially free of fluorine compounds.

7. The introducer sheath according to claim 1, wherein the inner lining has an inner diameter in a range of 1.3-12 mm.

8. The introducer sheath according to claim 1, wherein the inner lining has a wall thickness in a range of 0.1-0.3 mm.

9. The introducer sheath according to claim 1, wherein the outer casing comprises a polyether block amide copolymer.

10. The introducer sheath according to claim 1, wherein the outer casing has a wall thickness in a range of 0.1-0.3 mm.

11. The introducer sheath according to claim 1, wherein the reinforcing layer comprises a braided metal strip and/or a spiral metal strip.

12. The introducer sheath according to claim 11, wherein the metal strip has a thickness in a range of 0.2-0.5 mm.

13. A method for manufacturing an introducer sheath according to claim 1, comprising the following method steps:
a. providing the inner lining, the outer casing, and the reinforcing layer;
b. arranging the inner lining around a mandrel;
c. arranging the reinforcing layer around the inner lining;
d. arranging the outer casing around the reinforcing layer;
e. encasing the outer casing with a shrink tube to form a structure;
f. heating the structure comprising the inner lining, the reinforcing layer, the outer casing, and the shrink tube while shrinking the shrink tube and while joining with a material bond the inner lining to the outer casing by means of the reinforcing layer;
g. removing the mandrel.

14. The method according to claim 13, wherein the provision of the inner liner comprises extruding a granulate mixture comprising the thermoplastic elastomer and the siloxane.

15. The method according to claim 14, wherein the granulate mixture comprises a first granulate consisting of the thermoplastic elastomer and a second granulate comprising the siloxane in a matrix of the thermoplastic elastomer.
